SEQUENCE LISTING (8 sequences)

SEQ ID NO: 1 - DNA, 21 bp, mus musculus
ctggagctgcccgggccagcc

SEQ ID NO: 2 - DNA, 20 bp, mus musculus
caaggctgaccacatctggg

SEQ ID NO: 3 - DNA, 22 bp, mus musculus
ccactccgattaccggcgcatc

SEQ ID NO: 4 - DNA, 20 bp, mus musculus
gctcctgcagcggccgcacg

SEQ ID NO: 5 - DNA, 20 bp, mus musculus
aagacgtcagctggaccgac

SEQ ID NO: 6 - DNA, 22 bp, mus musculus
tcttgttggtaaaggcagtccc

SEQ ID NO: 7 - DNA, 20 bp, mus musculus
accagcgccacttcttcacg

SEQ ID NO: 8 - DNA, 20 bp, mus musculus
gtggagcatcatactgatcc

(19) United States
(12) Patent Application Publication
(10) Pub. No.: US 2004/0087530 A1
(43) Pub. Date: May 6, 2004

(54) Methods and compositions for modulating leptin activity

(75) Inventors: Jeffrey S. Flier, West Newton, MA (US); Christian Bjorbaek, Boston, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Beth Israel Deaconess Medical Center, Inc., Boston, MA

(21) Appl. No.: 10/430,683

(22) Filed: May 6, 2003

Related U.S. Application Data
(63) Continuation of application No. 09/637,247, filed on Aug. 11, 2000, now abandoned.
Application No. 09/637,247 is a continuation of application No. PCT/US99/02865, filed on Feb. 10, 1999.
Application No. PCT/US99/02865 is a continuation-in-part of application No. 09/044,278, filed on Mar. 19, 1998, now abandoned.
(60) Provisional application No. 60/074,320, filed on Feb. 11, 1998.

(51) Int. Cl. A61K 48/00; A61K 39/395

(52) U.S. Cl. 514/44; 424/145.1

(57) ABSTRACT

Administration of leptin affects food intake and body weight in animals and humans by a mechanism involving actions on specific regions of the hypothalamus. CIS-1, SOCS-1, SOCS-2 and SOCS-3 genes were investigated for their ability to antagonize leptin action. In mammalian cell lines, SOCS-3 completely blocked leptin induced signal- transduction, whereas CIS, SOCS-1 and SOC-2 were without effect. SOCS-3 is a major target of leptin action in leptin responsive cells in the hypothalamus, and SOCS-3 is a potent inhibitor of leptin signaling. Increased SOCS-3 activity in leptin-responsive neurons is a potential mechanism for the leptin resistance observed in syndromes of obesity, affective mood disorders and reproductive disorders.

### RELATED APPLICATIONS

This application is a Continuation of U.S. patent application Ser. No. 09/637,247, filed Aug. 11, 2000, which is a continuation of and claims priority to International Application No. PCT/US99/02865, filed Feb. 10, 1999, which is a Continuation-in-Part of and claims priority to U.S. patent application Ser. No. 09/044,278 filed Mar. 19, 1998 which claims the benefit of U.S. Provisional Application No. 60/074,320 filed Feb. 11, 1998, the teachings of which are incorporated herein by reference in their entirety.

### BACKGROUND OF THE INVENTION

Leptin, the adipocyte derived hormone, acts on specific regions of the brain to regulate food intake, energy expenditure and neuroendocrine function (Zhang Y, et al., Nature 372:425-432, 1994; Halaas J L et al., Science 269:543-546, 1995; Campfield L A, et al., Science 269:546-549,1995, and Pellymounter M A, et al., Science 269:540-543, 1995.). Leptin is structurally related to cytokines (Zhang F, et al., Nature 387:206-209, 1997) and acts on receptors that belong to the cytokine-receptor superfamily (Tartaglia L A, et al., Cell 83:1263-1271; Lee G -H, et al., Nature 379:632-635, 1996).

In diet-induced obesity in rodents, and in most humans with obesity, resistance to peripheral leptin exists, and has yet to be explained. Human obesity could be related to low levels of functional circulating leptin or to decreased action at the target cells in the brain. Supporting the latter possibility are data demonstrating that while functional mutations in the leptin gene exist in humans (Montague C T, et al., Nature 387:903-908, 1997), they are extremely rare (Maffei M, et al., Diabetes 45:679-682, 1996; Shigemoto M, et al., Eur J Endocrinol. 13 7:511-513, 1997; Carlsson B, et al., Obes Res 5:30, 1997). In addition, serum leptin levels are increased in human obesity and correlate positively with body weight (Maffei Metal., Nat Med. 1:1155-1161, 1995; Considine R V et al., Engl J Med. 334:292-295, 1996). Furthermore, some (Widdowson P S et al., Diabetes 46:1782-1785, 1997), but not all studies (Van Heek M et al., J Clin Invest 99:385-390, 1997) of diet-induced obesity in rodents show that these animals develop both peripheral and central resistance to recombinant leptin. Together, these data are consistent with the possibility that the leptin-resistance which characterizes human obesity may be due to defects in leptin signal-transduction in the brain. Two potential mechanisms for leptin resistance are defects at the level of the blood brain barrier, and defects in the pathway of leptin signal transduction in target cells. Regarding the latter possibility, the leptin receptor (OBR) (mutation of which causes obesity in db/dh mice and fa/fa rats), is most closely related to the gp130 and LIFR signal transducing subunits that are activated by cytokines such as IL-6, LIF and CNTF and hormone receptors for growth hormone such as erythropoietin (Tartaglia, 1995). Several isoforms of the leptin receptor exist including a long form that is predominantly expressed in specific cell bodies in the hypothalamus. Potential mechanisms for inhibiting or enhancing leptin signaling are a matter of considerable interest.

Recently, a new family of cytokine-inducible inhibitors of signaling has been identified including CIS (cytokine-inducible sequence), SOCS-1 (suppressor of cytokine signaling), SOCS-2 and SOCS-3 (Starr R et al, Nature 387:917-921, 1997; Endo T A, et al, Nature 387:921-924, 1997; Naka T et al, Nature, 387:924-929, 1997; Masuhara M et al, Biochem Biophys Res Commun, 239:439-446, 1997). A number of different cytokines including IL-6, LIF, growth hormone (GH) and erythropoietin (EPO) induce transcriptional activation of one or more of the CIS or SOCS genes in vivo and in vitro, through activation of the JAK-STAT pathway (Starr, 1997; Endo, 1997; Naka, 1997; Yoshimura et al. Embo J. 14:2816-2826, 1995, Masuhara, 1997). The results suggest that the CIS and SOCS proteins may act in a classic negative feedback loop by inhibiting JAK activity, thereby switching off cytokine signal transduction.

### SUMMARY OF THE INVENTION

The present invention encompasses methods and compositions for altering, or modulating, leptin activity by altering, or modulating, cytokine inhibitor activity. Specifically encompassed in the present invention are methods and compositions to alter activity of the cytokine inhibitor, SOCS-3. SOCS-3 expression is rapidly induced by leptin treatment in regions of the hypothalamus that are known to be involved in the regulation of body weight. As demonstrated herein, it has now been determined that a SOCS-3-mediated leptin cell-signaling inhibitory pathway exists. Thus, this suggests that SOCS-3 is a negative regulator of leptin signal-transduction. Also as described herein, it is believed that excessive SOCS-3 activity is an important factor in the leptin resistance that characterizes most syndromes of rodent and human obesity. Inhibition of SOCS-3 expression or function is therefore a potential target for the development of drugs aimed at improving leptin sensitivity in a mammal and hence inducing weight loss. Furthermore, inappropriately increased SOCS-3 activities in leptin-responsive neurons is a potential mechanism for the leptin resistance observed in various syndromes of obesity. Thus, as described herein, altering SOCS-3 activity provides a means for modulating leptin-induced cell signaling and therefore modulating bodyweight.

The present invention also relates to methods of treating delayed onset of puberty in mammals by increasing SOCS-3-mediated leptin cell signaling and to methods of treating reproductive dysfunction, or infertility, such as anovulation or decreased spermatogenesis associated with low serum/plasma levels of leptin, inactive leptin, leptin resistance or ineffective production of leptin. The methods of the present invention can be used in either male or female mammals.

The present invention also relates to the methods of treating affective mood disorders in an individual associated with elevated leptin levels, such as atypical depression, wherein atypical depression is characterized by elevated leptin levels in an individual such that treatment resulting in decreased leptin-induced cell signaling would result in prevention or alleviation of symptoms of atypical depression. The present invention also relates to methods of treating affective mood disorders associated with decreased leptin levels, such as melancholic depression, wherein melancholic depression is characterized by decreased leptin levels in an individual, such that treatment resulting in increased leptin-induced cell signaling would result in prevention or alleviation of symptoms of melancholic depression.

The present invention therefore pertains to methods of modulating leptin cell signaling by altering SOCS-3 activity in a mammal. As defined herein, modulating (also referred to herein as altering, adjusting or regulating) leptin activity means inhibiting or enhancing the biological activity of leptin or SOCS-3. Inhibiting leptin activity encompasses partial inhibition as well as complete abrogation of leptin activity.

The biological activity of leptin is defined herein as the ability of leptin to activate one, or more signal transduction pathways in a cell as a result of interaction between (e.g., binding) leptin and a leptin receptor associated with the cell. The signal transduction pathway includes for example, the activation of Janus Kinase 2 (referred to herein as JAK2), thereby activating other pathways, such as signal transducers and activators of transcription (STAT), phosphoinositide-3 kinase ras/mitogen-activated protein kinase pathways ultimately leading to activation or inactivation of gene transcription as well as other, non-transcriptional effects. For example, leptin can bind its cognate receptor, which is associated with JAK2; JAK2 is activated, and phosphorylates the receptor, JAK2 and STAT3 proteins (among others). Phosphorylated STAT3 dimerizes and translocates to the nucleus, where it serves as a transcriptional activator. Therefore, leptin activity can be measured as the level of phosphorylation of the receptor, JAK2 or STAT3. Further, leptin activity can be measured by the amount of gene transcription from STAT3 responsive genes.

As defined herein, SOCS-3 activity, or SOCS-3 mediated leptin cell signaling, is the inhibition or inactivation (completely or partially) of leptin induced cell signaling. As demonstrated by the present invention, SOCS-3 mediates the down regulation of leptin signaling as measured by lack of phosphorylation of leptin receptor, JAK2 or STAT3, as well as by the association of JAK2 and SOCS-3. As described herein, SOCS-3 transcription is part of a negative feedback loop triggered by leptin activation of the leptin receptor.

SOCS-3 activity can be inhibited by inhibiting or reducing the amount of SOCS-3 protein expressed in a cell, or by introducing a polynucleotide encoding a modified SOCS-3 protein into a cell, wherein the modified SOCS-3 protein comprises a mutant, variant, derivative, or analog of the SOCS-3 protein.

SOCS-3 expression can be inhibited or reduced by transfecting a cell with a polynucleotide construct encoding SOCS-3 antisense DNA or RNA. For example, the antisense RNA can hybridize to the endogenous SOCS-3 mRNA and prevent translation of SOCS-3 mRNA, thereby inhibiting or reducing expression of SOCS-3 protein. SOCS-3 expression can also be inhibited or reduced by transfecting the cell with a polynucleotide construct encoding a transcriptional inhibitor such that transcription of SOCS-3 is inhibited or reduced. Such a transcriptional inhibitor would interact specifically with SOCS-3 promoter sequences, resulting in decreased transcription of SOCS-3, decreased SOCS-3 protein expression and thus decreased SOCS-3 activity.

SOCS-3 activity can also be inhibited by transfecting the cell with a polynucleotide construct encoding an altered, or modified SOCS-3 protein, polypeptide or peptide. In one embodiment, the modified SOCS-3 polypeptide is a competitive inhibitor (e.g., antagonist) of endogenous SOCS-3. The modified SOCS-3 can interact with a SOCS-3 target protein (e.g., JAK2), without interfering with the activity of the target protein. Because the modified SOCS-3 protein interacts with the intended SOCS-3 target, endogenous SOCS-3 could not interact with its intended target, thereby inhibiting or reducing the level of SOCS-3 mediated leptin cell signaling. In another embodiment, SOCS-3 activity can be inhibited or reduced by introducing a SOCS-3 inhibitor into the cell. Such an inhibitor can be a peptide or small organic molecule that interferes with SOCS-3 activity. Such an inhibitor can interact specifically with SOCS-3, or to its intended target, to inhibit SOCS-3 activity. For example, the inhibitor can interact with downstream targets of SOCS-3 such as JAK2.

The present invention further encompasses methods of increasing or enhancing SOCS-3 activity in a cell. Increased SOCS-3 activity in a cell can inhibit or reduce leptin-induced cell signaling. A reduction or inhibition of leptin-induced cell signaling can be useful to prevent, inhibit or alleviate atypical depression in an individual, or to promote weight gain in an individual. SOCS-3 activity can be increased by transfecting a cell with a polynucleotide construct encoding a biologically active form of SOCS-3 protein, or a biologically active fragment thereof. In another embodiment, SOCS-3 activity can be increased by transfecting a cell with a nucleic acid encoding a modified SOCS-3 protein that has increased biological activity.

The present invention also pertains to cell lines that can be used to evaluate SOCS-3 mediated leptin activity and to screen candidate SOCS-3 inhibitors, antagonists and agonists for activity. For example, a cell line can be produced that expresses SOCS-3, a cytokine receptor and a reporter gene construct wherein transcription of the reporter gene construct is inhibited by SOCS-3. In one embodiment, the cytokine receptor is the leptin receptor. In another embodiment, the reporter gene construct is a leptin responsive promoter attached to a reporter gene. The reporter gene can be the CAT gene, the luciferase gene or the β-galactosidase gene. Another cell line suitable for use in the present invention is a cytokine dependent cell line wherein SOCS-3 and the leptin receptor are stably expressed. In one embodiment, the cytokine receptor is the IL-3 receptor.

The cell lines of the present invention can be used to screen libraries such as organic molecule libraries or cDNA libraries to select and identify molecules that inhibit (or enhance) SOCS-3 activity. In one embodiment, cells expressing the leptin receptor, SOCS-3 and a reporter gene construct are contacted with an organic molecule library or transfected with a cDNA expression library. These cells are then stimulated with leptin. Cells having increased reporter gene activity are selected and the organic molecule or cDNA is identified. In another embodiment, IL-3 dependent cells expressing leptin receptor and SOCS-3 are removed from IL-3, contacted with a member of an organic molecule library or transfected with a member of a CDNA expression library. Cells capable of proliferating in leptin are selected and the organic molecule or cDNA is identified.

Thus, as a result of the discovery described herein, methods and compositions are now available to modulate leptin activity, specifically by modulating the activity of the cytokine inhibitor, SOCS-3, thereby resulting in either an increase or decrease of leptin-induced cell signaling.

### BRIEF DESCRIPTION OF THE DRAWINGS

FIG. 1A shows the results of a ³²P-RT-PCR assay demonstrating the in vivo effects of leptin on CIS-1, SOCS-1, SOCS-2, and SOCS-3 mRNA levels in the hypothalamus from ob/ob mice.

FIG. 1B is the quantification of the data in FIG. 1A.

FIG. 1C is a plot of quantitative ³²P-RT-PCR of SOCS-mRNA from hypothalami of db/db mice and lean (+/?) controls upon leptin treatment.

FIGS. 2A and 2B show the results of in situ hybridization with ³⁵S-labeled antisense SOCS-3 probes to brain sections from normal rats treated with saline or leptin.

FIG. 3A is a graphic representation of leptin-induced erg-1 promoter activation by SOCS-3 in CHO cells.

FIG. 3B shows a Western blot demonstrating inhibition of leptin induced leptin receptor tyrosine phosphorylation by SOCS-3 in COS-1 cells.

### DETAILED DESCRIPTION OF THE INVENTION

The present invention encompasses the regulation of leptin activity in the brain. Specifically encompassed by the present invention is the regulation of the leptin-induced cell signaling pathway in the hypothalamus via regulation of SOCS-3 activity.

Leptin is a hormone which has been shown to interact with its cognate receptor, thereby initiating its cell-signaling pathway. Several different leptin receptor isoforms are predicted to exist, including a long form which has the highest level of expression in regions of the hypothalamus, and specific regions therein, including the arcuate nucleus and the dorso-medial hypothalamus. In vitro and in vivo studies demonstrate that leptin activates cytokine-like signal transduction by stimulating the classic JAK-STAT pathway via the long receptor isoform (Ghilardi N et al, Proc Natl Acad Sci USA. 93, 6231-6235, 1997; Baumann H et al, Proc Natl Acad Sci USA. 93:8374-8378, 1996; Vaisse C, et al, Nature Genetics 14:95-97, 1996). Lack of functional leptin or of long form leptin receptors in the ob/ob and db/db mice, respectively, causes severe obesity (Zhang et al, Nature 372:425-432, 1994; Lee G -H, et al, Nature 379:632-635, 1996; Chen H et al, Cell 84:491-495, 1996).

The cloning of the genes encoding leptin (Zhang, 1994,) and the leptin receptor (Tartaglia et al., 1995, 61183;1263-1271), and the study of these proteins in vivo and in vitro, have dramatically demonstrated the importance of this ligand-receptor system in the normal regulation of body weight and energy balance.

In addition to this role which has been proposed to be its primary function, circulating leptin also appears to play an important role in the neuroendocrine axis (Ahima, R. S., et al., Nature 382:250-252, 1996), including the regulation of reproduction. Administration of exogenous leptin has been shown to induce the onset of puberty in mice (U.S. patent application Ser. No. 08/749,534, the teachings of which are incorporated herein by reference in its entirety). Treatment of ad lib. fed female mice with a dose of leptin which did not significantly alter body weight, resulted in an earlier onset of puberty.

Recently, a new family of cytokine inhibitors has been described, including CIS-1 and SOCS-1, 2, and 3. CIS-1, an inhibitor of cytokine receptor signaling, is thought to bind directly to the cytokine receptors and possibly block key phosphotyrosine residues of the receptor (Yoshimura, 1995). SOCS-1, 2 and 3 are cytokine inducible inhibitors that were found to be active in hematopoietic cells (Starr, 1997; Endo, 1997; and Naka,1997) where it is thought to abrogate cytokine mediated cell proliferation. As demonstrated herein for the first time, leptin specifically induces expression of SOCS-3 mRNA in hypothalamic nuclei known to express high levels of the long form of the leptin receptor. No effect on CIS, SOCS-1 or SOCS-2 could be detected. (See Example 1).

In situ hybridization experiments with SOCS-3 antisense RNA probes to brain sections from both ob/ob mice and normal rats demonstrated a leptin dependent specific increase of SOCS-3 mRNA in the arcuate nucleus and dorso-medial hypothalamus (Example 2). These regions express the highest level of the long form leptin receptor mRNA in the hypothalamus, strongly suggesting that the effect by leptin on SOCS-3 mRNA levels is a direct effect in specific neurons expressing long form leptin receptors. The regions of the arcuate nucleus that show stimulation of SOCS-3 mRNA after leptin treatment are regions known to express NPY, POMC and AGRP, all of which are regulated by leptin in vivo, suggesting that cells expressing these neuropeptides may be direct targets of leptin.

Fos is often used as a marker for activated neurons. However, its use is limited due to the inability to distinguish between direct and indirect actions of a given agent. Furthermore, NPY-expressing neurons in the arcuate nucleus, which are negatively regulated by leptin, are not positive for Fos activation after leptin treatment. However, as demonstrated in Example 2, herein, SOCS-3 mRNA is increased in the region of the arcuate nucleus which express NPY, suggesting that SOCS-3 is a better marker than Fos for neurons that are regulated by leptin.

The lethal yellow (A^{y}/a) mouse develops obesity due to ectopic and unregulated overexpression of the agouti protein (Dickie, J. Hered. 60:20-25, 1969; Bultman et al., Cell 71:1195-1204, 1992; Miller et al., Genes Dev. 7:454-467, 1993), a potent melanocortin receptor (MCR) antagonist. The obesity in this model is characterized by hyperleptinemia and by resistance to both central and peripheral leptin administration (Halaas, 1997). Ultimately, the leptin resistance in this model must result from melanocortin antagonism induced by agouti, but the molecular basis for the leptin resistance is unknown. It has been hypothesized that the leptin resistance of A^{y}/a mice may be due to the blockade of MC4 receptors at a site in the brain downstream of leptin signaling (Seeley et al., Nature 390:349, 1997). However, it has recently been demonstrated that A_{y}/a mice that are also deficient in leptin, e.g. A^{y}/ a, lep^{ob}/lep^{ob}, are normally responsive to leptin (Boston et al., Science 278:1641-1644, 1997). The latter observation suggests that leptin resistance in A^{y}/a mice is a result of, or at least requires, in addition to MC4 receptor blockade, chronic exposure to high circulating levels of leptin. As described in Example 4, in situ hybridization histochemistry revealed that SOCS-3 mRNA is elevated in ad libitum fed A^{y}/a mice as compared to lean control litter mates and in particular, in the dorsomedial hypothalamic nucleus, a site in which leptin induces SOCS-3 gene expression. A similar region of the dorsomedial hypothalamic nucleus contains Fos-like immunoreactivity following intravenous (Elmquist et al., Endocrinology 138:839-842, 1998) or central leptin administration to normal rats (Van Dijk, et al., Am J Physiol., 271:R1096-R1100, 1996). This site has also been demonstrated to have increased levels of NPY mRNA in A^{y}/a mice (Kesterson et al., Mol Endocrinol. 11:360-637, 1997). Thus it is reasonable to believe that leptin resistance in this model is a consequence of hyperleptinemia; serum leptin levels in the A^{y}/a mice employed here were 40 ng/ml compared to 7 ng/ml in control mice. In this model, increased leptin could drive SOCS-3 expression in key hypothalamic nuclei involved in body weight regulation, thereby inhibiting the weight reducing effects of leptin.

In mammalian cell lines, SOCS-3, but not CIS-1, or SOCS-2, completely blocked leptin induced signal- transduction (described in Example 5), suggesting that leptin-receptor signaling is also negatively regulated by SOCS-3 in vivo.

It is thought that the leptin receptor long form may exert a signaling action similar to that of granulocyte colony stimulating factor, leukemia inhibitory factor receptor and gp130. Ligand binding to these receptors leads to activation of receptor-bound JAK kinases, which phosphorylate tyrosines in the cytoplasmic domain of the receptor as well as in other cytoplasmic target proteins. Several pathways can be activated by JAK kinases, including the signal transducers and activators of transcription (STAT), ras/mitogen-activated protein kinase, and phospho-inositide-3 kinase pathways. As shown in Example 7, leptin stimulation in vitro results in the induction of SOCS-3 mRNA. In addition, when SOCS-3 is present, leptin-induced phosphorylation of STAT3, JAK2 as well as the leptin receptor is inhibited. Furthermore, leptin pretreatment of CHO cells expressing the leptin receptor resulted in a prolonged inhibition (longer than 24 hours) of subsequent leptin induced signaling. For example, as described in Example 8, SOCS-3 mRNA was not induced, STAT3 DNA binding activity was not increased nor was the leptin receptor phosphorylated in response to subsequent leptin treatment. This inhibition lasted for at least 24 hours after leptin pretreatment.

Based on the results described herein, it is reasonable to believe that SOCS-3 antagonizes leptin induced cell-signaling by interacting with JAK2 and competing with binding between JAK2 and its substrate (leptin receptor or STAT) or by acting as a JAK2 pseudo substrate, thereby preventing phosphorylation of the intended target. SOCS-3 can inhibit JAK2 kinase activity thereby preventing phosphorylation of downstream elements. Thus, it has now been determined that a SOCS-3-mediated leptin cell-signaling inhibitory pathway exists. SOCS-3 therefore, can negatively regulate cell signaling via the leptin receptor.

SOCS-3 might antagonize leptin-induced cell signaling by recruitment of tyrosine-phosphatases, which have been shown to be involved in dephosphorylation of the cytokine receptors. Two candidates are SHP-1 and SHP-2 (also known as SYP). Both SHP-1 and 2 have been shown to regulate cytokine signaling. Recruitment of SHP-1 has been associated with dephosphorylation/inactivation of JAK2 and subsequent termination of erythropoietin signal transduction (Klingmüller, et al, Cell 80:729-738, 1995). A similar role for SHP-1 in mediating the down-regulation of JAK2 following stimulation of cells with growth hormone has been proposed.

The present invention encompasses methods and compositions for modulating leptin activity comprising altering SOCS-3 activity in a cell, for example, a hypothalamic cell. Cells encompassed by the present invention can be found in all vertebrates including mammals and humans. The cells could also be cells maintained in a cell line, e.g., transformed cells which are suitable for use in testing leptin or SOCS-3 activity.

In one embodiment of the present invention, it is desirable to increase, or up-regulate leptin activity via inhibition of SOCS-3 activity. Increasing leptin activity results in the increase of the leptin cell-signaling pathway, resulting in, inter alia, weight loss, restoration of reproductive function and/or alleviation of the symptoms of melancholic depression. In this embodiment, SOCS-3 activity is inhibited by interfering with the interaction (e.g., binding) of SOCS-3 to its intended target. For example, the intended target of SOCS-3 interacts with JAK2 (Example 6). SOCS-3 may act as a pseudo-substrate of JAK2, or may recruit phosphatases to the JAK2-receptor complex. Thus, a polypeptide or peptide inhibitor/antagonist comprising the SOCS-3 amino acid sequence of GenBank Accession Number U88328, or a modified SOCS-3 amino acid sequence, or an active fragment thereof, can competitively interact with SOCS-3 and/or its intended target, e.g. by binding to JAK2, resulting in the increase or up-regulation of leptin activity.

As defined herein, modified SOCS-3 encompasses SOCS-3 molecules comprising fragments, derivatives, analogs, variants and mutants of the SOCS-3 protein. These modified SOCS-3 molecules possess SOCS-3 inhibitor/antagonist activity, thereby inhibiting the activity of endogenous SOCS-3 present in a cell, resulting in an increase of leptin activity. Another activity of modified SOCS-3 molecules can be the antigenic property of the modified SOCS-3 molecule comprising the ability of the modified SOCS-3 to bind to SOCS-3-specific antibodies. The modified SOCS-3 molecule can also possess immunogenic properties whereby the modified SOCS-3 molecule induces an immunogenic response, e.g., the production of antibodies that specifically bind to endogenous (native) SOCS-3.

A fragment of SOCS-3 encompasses polypeptides that comprise only a part of the full-length SOCS-3 protein and inhibit endogenous SOCS-3 activity. Such fragments can be produced by amino and/or carboxyl terminal deletions, as well as internal deletions. Fragments can also be produced by enzymatic digestion. Such modified SOCS-3 molecules can be tested for inhibitory activity as described herein.

“Derivatives” and “variants” of SOCS-3 can include truncated and hybrid forms of SOCS-3. “Truncated” forms are shortened forms of SOCS-3, typically with internal deletions of regions of the protein. “Hybrid” forms of SOCS-3 are SOCS-3 molecules comprising a portion of a SOCS-3 amino acid sequence with non-SOCS-3 amino acid sequence, e.g., SOCS-1 or SOCS-2 sequence.

“Variants” and “mutants” of SOCS-3 can be produced using in vitro and in vivo techniques well-known to those of skill in the art, for example, site-specific mutagenesis and oligonucleotide mutagenesis. Manipulations of the SOCS-3 protein sequence can be made a the protein level as well. Any numerous chemical modifications can be carried out by known techniques including, but not limited to, specific chemical cleavage by cyanogen bromide, trypsin and papain. SOCS-3 can also be structurally modified or denatured, for example, by heat. In general, mutations can be conservative or non-conservative amino acid substitutions, amino acid insertions or amino acid deletions. The mutations can be at or near SOCS-3 binding sites.

For example, DNA encoding a SOCS-3 mutant is prepared by site-directed mutagenesis of DNA that encodes endogenous SOCS-3. Site-directed (site-specific) mutagenesis allows the production of SOCS-3 variants through the use of specific oligonucleotide sequences that encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 20 to 25 nucleotides in length is preferred, with about 5 to 10 residues on both sides of the junction of the sequence being altered. In general, the techniques of site-specific mutagenesis are well known in the art, as exemplified by publications such as Edelman et al., DNA 2:183, 1983. The site-specific mutagenesis technique typically employs a phage vector that exists in both a single-stranded and double-stranded form. Typical vectors useful in site-directed mutagenesis include vectors such as the M13 phage, for example, as disclosed by Messing et al., Third Cleveland Symposium on Macromolecules and Recombinant DNA, A. Walton, ed., Elsevier, Amsterdam, 1981. This and other phage vectors are commercially available and their use is well-known to those skilled in the art. A versatile and efficient procedure for the construction of oligonucleotide directed site-specific mutations in DNA fragments using M13-derived vectors was published by Zoller, M. J. and Smith, M., Nucleic Acids Res. 10:6487-6500, 1982. Also, plasmid vectors that contain a single-stranded phage origin of replication can be employed to obtain single-stranded DNA, Veira et al., Meth Enzymol. 153:3 1987.

Alternatively, nucleotide substitutions can be introduced by synthesizing the appropriate DNA fragment in vitro, and amplifying it by PCR procedures known in the art.

In general, site-specific mutagenesis herewith can be performed by first obtaining a single-stranded vector that includes within its sequence a DNA sequence that encodes the relevant protein. An oligonucleotide primer bearing the desired mutated sequence is prepared, generally synthetically, for example, by the method of Crea et al., Proc Natl Acad Sci USA. 75:5765, 1978. This primer can then be annealed with the single-stranded protein sequence-containing vector, and subjected to DNA polymerizing enzymes such as E. coli polymerase I Klenow fragment, to complete the synthesis of the mutation-bearing strand. Thus, a heteroduplex is formed wherein one strand encodes the original non-mutated sequence and the second strand bears the desired mutation. This heteroduplex vector can then be used to transform appropriate host cells such as JM 101 cells, and clones can be selected that include recombinant vectors bearing the mutated sequence arrangement. Thereafter, the mutated region can be removed and placed in an appropriate expression vector for protein production.

The PCR technique can also be used in creating amino acid sequence variants of SOCS-3. When small amounts of template DNA are used as starting material in a PCR, primers that differ slightly in sequence from the corresponding region in a template DNA can be used to generate relatively large quantities of a specific DNA fragment that differs from the template sequence only at the positions where the primers differ from the template. For introduction of a mutation into a plasmid DNA, one of the primers can be designed to overlap the position of the mutation and to contain the mutation; the sequence of the other primer is preferably identical to a stretch of sequence of the opposite strand of the plasmid, but this sequence can be located anywhere along the plasmid DNA. It is preferred, however, that the sequence of the second primer is located within 500 nucleotides from that of the first, such that in the end the entire amplified region of DNA bounded by the primers can be easily sequenced. PCR amplification using a primer pair like the one just described results in a population of DNA fragments that differ at the end position of the mutation specified by the primer.

The DNA fragments produced bearing the desired mutation can be used to replace the corresponding region in the plasmid that served as PCR template using standard DNA technology. Mutations at separate positions can be introduced simultaneously by either using a mutant second primer or performing a second PCR with different mutant primers and ligating the two resulting PCR fragments simultaneously to the vector fragment in a three (or more) part ligation.

Another method for preparing variants, cassette mutagenesis, is based on the technique described by Wells et al. Gene 34, 315, 1985. The starting material can be the plasmid (or vector) comprising the SOCS-3 DNA to be mutated. The codon(s) within the SOCS-3 to be mutated are identified. There must be unique restriction endonuclease sites on each side of the identified mutation site(s). If such restriction sites do not exist, they can be generated using the above-described oligonucleotide-mediated mutagenesis method to introduce them at appropriate locations in the SOCS-3 DNA. After the restriction sites have been introduced into the plasmid, the plasmid is cut at these sites to linearize it. A double stranded oligonucleotide encoding the sequence of the DNA between the restriction sites but containing the desired mutation(s) is synthesized using standard procedures. The two strands are synthesized separately and then hybridized together using standard techniques. This double-stranded oligonucleotide is referred to as the cassette. This cassette is designed to have 3′ and 5′ ends that are compatible with the ends of the linearized plasmid, such that it can be directly ligated to the plasmid. The plasmid now contains the mutated SOCS-3 DNA sequence, that can be expressed to produce SOCS-3 with altered binding activity.

The inhibitor compounds of the present invention include any molecule that interacts with endogenous SOCS-3 or to SOCS-3 target molecules such as JAK2 such that upon interacting with said molecules, inhibitors the SOCS-3 mediated inhibition of leptin cell-signaling activity. Encompassed by the present invention are inhibitor compounds that mimic the structure and conformation of the substrate moiety when interacting with the binding or active site. Molecular inhibitors of the present invention will typically have an inhibition constant (Kᵢ) of ten micromolar, or less. Specifically encompassed are organic molecules that mimic the structure and conformation of SH2 binding domains and interact with SOCS-3, thereby inhibiting its activity. In one embodiment the inhibitor contains or mimics phosphotyrosine.

Also encompassed by the present invention are small organic molecules that mimic the structure of SOCS-3, or, alternatively, the binding site of the SOCS-3 target, and therefore, interfere with the interaction of SOCS-3 with its intended target molecule.

Peptides suitable for use as SOCS-3 inhibitors can be produced in libraries. The peptides of the library can be immobilized on a surface, for example the peptides can be immobilized on a chip or on beads.

The libraries of peptides comprise a mixture of substantially equimolar amounts of peptides. In one embodiment, the library can be designed to mimic SOCS-3 target molecules, e.g., JAK2. In another embodiment, the library comprises peptides or phostyrosin containing peptides that interact with the SH2 domain of SOCS-3, thereby inhibiting the ability of SOCS-3 to bind target molecules.

The inhibitors of the present invention can be synthesized using standard laboratory methods that are well known to those of skill in the art, including standard solid phase techniques. Inhibitors comprising naturally occurring amino acids can also be produced by recombinant DNA techniques known to those of skill, and subsequently phosphorylated.

The inhibitors of the present invention can comprise either the 20 naturally occurring amino acids or other synthetic amino acids. Synthetic amino acids encompassed by the present invention include, for example, naphthylalanine, L-hydroxypropylglycine, L-3,4-dihydroxyphenylalanyl, α-amino acids such as L-α-hydroxylysyl and D-α-methylalanyl, L-α-methyl-alanyl, β amino-acids such as β-analine, and isoquinolyl.

D-amino acids and other non-naturally occurring synthetic amino acids can also be incorporated into the inhibitors of the present invention. Such other non-naturally occurring synthetic amino acids include those where the naturally occurring side chains of the 20. genetically encoded amino acids (or any L or D amino acid) are replaced with other side chains of the 20 genetically encoded amino acids (or any L or D amino acid) are replaced with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogs in which the ring size of the proline residue is changed from 5 members to 4, 6, or 7 member can be employed.

As used herein, “lower alkyl” refers to straight and branched chain alkyl groups having from 1 to 6 carbon atoms, such as methyl, ethyl propyl, butyl and so on. “Lower alkoxy” encompasses straight and branched chain alkoxy groups having from 1 to 6 carbon atoms, such as methoxy, ethoxy and so on.

Cyclic groups can be saturated or unsaturated, and if unsaturated, can be aromatic or non-aromatic. Heterocyclic groups typically contain one or more nitrogen, oxygen, and/or sulphur heteroatoms, e.g., furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. The heterocyclic groups can be substituted or unsubstituted. Where a group is substituted, the substituent can be alkyl, alkoxy, halogen, oxygen, or substituted or unsubstituted phenyl. (See U.S. Pat. No. 5,654,276 and U.S. Pat. No. 5,643,873, the teachings of which are herein incorporated by reference).

Peptide mimetics that mimic the SOCS-3 protein can also be designed to inhibit SOCS-3 activity, thereby resulting in an increase of leptin activity. These mimetics can be designed and produced by techniques known to those of skill in the art. (See e.g., U.S. Pat. Nos. 4,612,132; 5,643,873 and 5,654,276, the teachings of which are herein incorporated by reference). These mimetics are based on the SOCS-3 sequence, and possess activity antagonistic to the biological activity of the corresponding peptide compound, but possess a “biological advantage” over the corresponding peptide inhibitor with respect to one, or more, of the following properties: solubility, stability, and susceptibility to hydrolysis and proteolysis.

Methods for preparing peptide mimetics include modifying the N-terminal amino group, the C-terminal carboxyl group, and/or changing one or more of the amino linkages in the peptide to a non-amino linkage. Two or more such modifications can be coupled in one peptide mimetic inhibitor. Examples of modifications of peptides to produce peptide mimetics are described in U.S. Pat. Nos. 5,643,873 and 5,654,276, the teachings of which are incorporated herein by reference. Peptide mimetic libraries can also be produced as described above.

Alternatively, the SOCS-3 inhibitor can be an antibody or antibody fragment that interacts with SOCS-3, thereby preventing SOCS-3 from interacting with downstream target molecules such as JAK2, or such that SOCS-3 interacts with JAK2 without interfering with JAK2 kinase activity. The term “antibody” is meant to encompass polyclonal antibodies, monoclonal antibodies (mAbs), chimeric antibodies (e.g., humanized antibodies) and antibody fragments that retain the biological activity of specific binding to SOCS-3, such as Fab, Fab′, F(ab′)2 and Fv. Also encompassed are single-chain antibodies (sFvs). These antibody fragments lack the Fc portion of an intact antibody, clear more rapidly from the circulation and can have less non-specific tissue binding than an intact antibody. These fragments are produced by well-known methods in the art, for example by proteolytic cleavage with enzymes such as papain (to produce Fab fragments) or pepsin (to produce F(ab′)2 fragments.

Polyclonal antibodies are heterogeneous populations of antibody molecules derived from the sera of animals immunized with an antigen. A monoclonal antibody (mAb) contains a substantially homogenous population of antibodies specific to antigens, which population contains substantially similar epitope binding sites. MAbs may be obtained by methods known to those skilled in the art. See, for example Kohler and Milstein, Nature 256:495-497, 1975; U.S. Pat. No. 4,376,110; Ausubel et al, eds., Current Protocols in Molecular Biology, Green Publishing Assoc. and Wiley Interscience, N.Y., 1987, 1992; and Harlow and Lane Antibodies: A Laboratory Manual Cold Spring Harbor Laboratory, 1988; Colligan et al, eds., Current Protocols in Immunology, Greene Publishing Assoc. and Wiley Interscience, N.Y., 1992, 1993; the contents of which references are incorporated entirely herein by reference. Such antibodies can be of any immunoglobulin class including IgG, IgM, IgE, IgA, and any subclass thereof. A hybridoma producing a mAb of the present invention can be cultivated in vitro, in situ, or in vivo. Production of high titers of mAbs in vivo or in situ makes this the presently preferred method of production.

Chimeric antibodies which include humanized antibodies, are molecules wherein different portions of which are derived from different animal species, such as those having variable regions derived from a murine mAb and a human immunoglobulin constant region. Chimeric antibodies are primarily used to reduce immunogenicity in application and/or to increase yields in production, for example. Chimeric antibodies and methods for their production are known in the art (Cabilly et al., Proc Natl Acad Sci USA 81:3273-3277, 1984; Morrison et al., Proc Natl Acad Sci USA 81:6851-6855, 1984; Boulianne et al., Nature 312:643-646, 1984; Cabilly et al., European Patent Application 125023 (published Nov. 14, 1984); Neuberger et al., Nature 314:268-270, 1985; Taniguchi et al., European Patent Application 171496 (published Feb. 19, 1985); Morrison et al., European Patent Application 1739494 (published Mar. 5, 1986); Neuberger et al., PCT Application WO 86/01533, (published Mar. 13, 1986); Kudos et al., European Patent Application 184187 (published Jun. 11, 1986); Sahagan et al., J Immunol. 137:1066-1074, 1986; Robinson et al., International Patent Publication #PCT/US86/02269 (published 7 May 1987); Liu et al., Proc Natl Acad Sci USA 84:3439-3443, 1987; Sun et al., Proc Natl Acad Sci USA 84:214-218, 1987; Better et al., Science 240:1041-1043, 1988; and Harlow and Lane Antibodies: A Laboratory Manual, Cold Spring Harbor Laboratory, 1988. These references are entirely incorporated herein by reference.

Typically, antibodies of the present invention are high affinity anti-SOCS-3 antibodies, and fragments or regions thereof, that have potent inhibiting and/or neutralizing activity in vivo against SOCS-3. Such antibodies can include those generated by immunization using purified recombinant SOCS-3 or peptide fragments thereof.

Methods for determining antibody specificity and affinity can be found in Harlow, et al., Antibodies: A Laboratory Manual, Cold spring Harbor Laboratory Press, Cold Spring Harbor, N.Y., 1988; Colligan et al., eds., Current Protocols in Immunology, Greene Publishing Assoc. and Wiley Interscience, N.Y., 1992, 1993; and Muller, Meth. Enzymol., 92:589-601 1983; which references are entirely incorporated herein by reference.

Further, SOCS-3 inhibitors/antagonists can function at the genetic level. Such antagonists include agents which decrease, inhibit, block or abrogate SOCS-3 expression, production or activity. Such an agent can be an antisense nucleic acid or sequence specific peptide nucleic acid. In addition, such an antagonist may interfere with SOCS-3 promoter activity. Further, such an antagonist can be a SOCS-3 mutant such as a mutant that functions as a competitive inhibitor which can be introduced and expressed in the cell where SOCS-3 activity is to be reduced. The mutant can be a full length derivative of SOCS-3 or fragments or derivatives of SOCS-3 as described above, such that expression of the mutant in a cell, inhibits the endogenous SOCS-3 activity. Such antagonists can be introduced into a cell by transfection, for example calcium phosphate precipitation or lipofection; or by infection with a virus or pseudovirus containing the desired construct, or by electroproration. Methods of introducing nucleic acid into a cell are well known in the art.

In another embodiment, the present invention encompasses introducing into a cell a nucleotide expression construct, wherein said construct encodes a modified form of SOCS-3. A modified form of SOCS-3 can include a dominant negative SOCS-3. Such a molecule can competitively bind the SOCS-3 target molecule without inactivating said target molecule (e.g., a dominant negative SOCS-3 would bind its target molecule, such as JAK2 and prevent endogenous SOCS-3 from binding, such that JAK2 remains phosphorylated, and/or such that JAK2 remains capable of phosphorlyating the appropriate downstream molecules, such as the cytokine receptor or STAT molecule.

Several vectors for use in such constructs are well known in the art. Furthermore, mechanisms of delivery of said constructs to an individual are well known in the art. For example, recombinant expression vectors which include synthetic or cDNA-derived DNA fragments encoding modified SOCS-3 molecules comprising DNA encoding a modified SOCS-3 protein operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation, as described in detail below. The ability to replicate in a host, usually conferred by an origin of replication, and a selection gene to facilitate recognition of transformants may additionally be incorporated. Operably linked indicates that components are linked in such a manner that expression of the DNA encoding a fusion protein is controlled by the regulatory elements. Generally, operably linked means contiguous.

Mammalian expression vectors may comprise non-transcribed elements such as an origin of replication, a suitable promoter and enhancer linked to the gene to be expressed, and other 5′ or 3′ flanking nontranscribed sequences, and 5′ to 3′ nontranslated sequences, such as necessary ribosome binding sites, a poly-adenylation site, splice donor and acceptor sites, and transcriptional termination sequences.

The transcriptional and translational control sequences in expression vectors to be used in transforming vertebrate cells may be provided by viral sources. For example, commonly used promoters and enhancers are derived from Polyoma, Adenovirus 2, Simian Virus 40 (SV40), and human cytomegalovirus. DNA sequence derived from the SV40 viral genome, for example, SV40 origin, early and late promoter, enhancer, splice, and polyadenylation sites may be used to provide the other genetic elements required for expression of a heterologous DNA sequence. The early and late promoters are particularly useful because both are obtained easily from the virus as a fragment which also contains the SV40 viral origin or replication (Fiers et al., Nature 273:113, 1978. Smaller or larger SV40 fragments may also be used, provided the approximately 250 bp sequence extending from the Hind III site toward the BgII site located in the viral origin or replication is included. Exemplary vectors can be constructed as disclosed by Okayama and Berg (Mol Cell Biol 3:280, 1983.

Preferred eukaryotic vectors for expression of mammalian DNA include pIXY321 and pIXY344, both of which are yeast expression vectors derived from pBC102.K22 (ATCC 67,255) and yeast.

In a further embodiment of the present invention, a method is provided to increase leptin induced signaling, wherein leptin-induced signaling results in the phosphorylation of STAT molecules, thereby increasing the amount of gene transcription of STAT-responsive genes.

In another embodiment of the present invention it is desirable to decrease or down-regulate leptin activity via increasing the activity of the SOCS-3 mediated leptin cell signaling pathway, thereby resulting in weight gain or prevention/alleviation of symptoms of atypical depression. SOCS-3 activity can be increased by introducing into a cell a nucleic acid construct expressing SOCS-3 or a biologically active fragment thereof. In this embodiment the SOCS-3 protein, or biologically active fragment of SOCS-3, comprises a SOCS-3 protein or fragment with biological activity comparable to the activity of endogenous SOCS-3, resulting in the negative regulation of leptin activity.

The present invention further provides methods to identify molecules that modulate the SOCS-3-mediated leptin cell-signaling pathway. Specifically encompassed by the present invention are methods to identify inhibitors/antagonists/agonists of SOCS-3 activity. Inhibitors of SOCS-3 activity can be identified and tested in in vitro assays and in ex vivo cell-based assays, as described herein. Candidates exhibiting the desired activity in vitro or ex vivo can be further evaluated in art-accepted animal models.

Candidate inhibitors, such as peptides, small organic molecules or derivatives of JAK2, can be evaluated for their ability to specifically interact with SOCS-3 in standard binding or capture assays known in the art. For example, SOCS-3 can be immobilized to a suitable surface (such as wells of a plastic microtiter plate or on beads) and contacted under physiological conditions to the peptide library, organic molecule library or JAK2 derivatives that have been labeled for subsequent detection. In another embodiment, the peptide or small organic molecule library; the antibody or antibody fragments or the target molecule or target molecule derivatives can be immobilized on a solid support and contacted with SOCS-3.

Peptide libraries, such as an oriented peptide library (Z. Songyang et al. Cell 72:767, 1993; can be screened for peptides that interact with SOCS-3. Peptide libraries and other small organic molecule libraries can also be screened using other assays known in the art, such as proximity assays or Biospecific Interaction Analysis (BIA). Biospecific Interaction Analysis (BIA) in real time can be performed to evaluate candidate molecules for their ability to bind SOCS-3. Surface plasmon resonance (SPR), which is the basis for BIA measurements, is an optical phenomenon arising in metal films under conditions of total internal reflection. The phenomenon produces a sharp dip in the intensity of reflected light at a specific angle. The position of this resonance angle depends on several factors, including the refractive index of the medium close to the non-illuminated side of the metal film. Refractive index is directly related to the concentration of dissolved material in the medium. By keeping other factors constant, SPR is used to measure changes in the concentration of macromolecules in a surface layer of solution in contact with a dextran-coated gold film. Using the BIAcore™ instrument from Pharmacia Biosensor AB, the association and dissociation rate constants for a peptide or organic molecule binding to SOCS-3 can be measured. Polypeptides peptides, peptide mimics or small organic molecules exhibiting higher association constants (Kₐ) have the greatest potential for ability to interact with SOCS-3 and inhibit SOCS-3 activity.

The present invention includes cell lines suitable for use in the screening methods described herein. In one embodiment, the cell line is a mammalian cell line such as CHO cells, Ba/F3 cells, HepG2 cells or H35-hepatoma cells, wherein said cells stably express a cytokine receptor and a reporter gene construct wherein the reporter gene construct is active in the absence of SOCS-3. The cell line is further modified by the introduction of SOCS-3 whereby the reporter gene construct is inhibited by SOCS-3 expression. In one embodiment the cytokine receptor is the leptin receptor long form. In another embodiment, the reporter gene encodes luciferase. In another embodiment, the reporter gene encodes β-galactosidase. In a further embodiment, the reporter gene construct contains SOCS-3 promoter elements.

In a preferred embodiment, the cell lines, cell signaling components (such as leptin receptors, JAK2), SOCS-3 are of human origin.

Candidate antagonists/agonists can be assessed for their ability to inhibit/enhance SOCS-3 activity, by their ability to allow reporter gene expression or cell proliferation of SOCS-3 expressing cells comprising the steps of: culturing the cells described above under conditions suitable for maintenance and growth; contacting said cells with the candidate molecule or an organic molecule library comprising SOCS-3 inhibitors or transfecting the cells with a CDNA expressing the candidate molecule with a cDNA expression library comprising DNA encoding candidate SOCS-3 inhibitors; contacting the cells with leptin; selecting the cells having increased reporter gene activity and identifying the organic molecule or cDNA that had contacted the cells selected. Methods of measuring gene transcript and enhancing or inhibition thereof are well known to those of skill in the art.

The present invention further encompasses a cytokine dependent cell line wherein the cells also stably express SOCS-3 and the leptin receptor long form. For example, the cytokine can be IL-3, IL-6 and other closely related cytokines. In one embodiment, the cytokine dependent cell line is Ba/F3 cells. In another embodiment, the cytokine is IL-3. The invention further provides a method of isolating and identifying inhibitors of SOCS-3, comprising the steps of culturing the cytokine-dependent cells described above in the presence of said cytokine under conditions suitable for maintenance and growth; removing said cells from the cytokine (in the case of BA/F3, the cytokine would be IL-3), contacting the cells with a candidate organic molecule or with a library comprising SOCS-3 inhibitor molecules or transfecting said cells with a cDNA expressing a SOCS-3 candidate inhibitor or a cDNA expression library comprising DNA encoding candidates SOCS-3 inhibitors; contacting said cells with leptin under conditions suitable for growth and maintenance of the cells; selecting cells capable of proliferating in the presence of leptin and identifying the organic molecule of cDNA that contacted the cells selected as described. Methods to transfect cells with cDNA expression libraries and subsequently isolate the cDNA are well known in the art. (Sambrook et al, Molecular Cloning).

Candidate inhibitors/agonists can further be evaluated in animal models. Animal models where SOCS-3 activity can be evaluated are known in the art, for example see Leibel et al., J. Biol. Chem. 272:319337-319340, 1997.

Inhibitors identified as described by the present invention can be useful to treat obesity or prevent weight gain in a mammal. Such molecules may also be useful to treat affective depression, such as melancholic depression, to induce the onset of puberty or to correct reproductive dysfunction. Alternatively, some affective disorders can be treated by decreasing leptin activity. For example a SOCS-3 agonist can be administered to an individual in need of such treatment.

The present invention further encompasses methods of reducing food intake in a mammal comprising increasing leptin cell-signaling comprising inhibiting SOCS-3 activity. In one embodiment, the mammal loses bodyweight.

The present invention further comprises a method of inducing puberty in a mammal comprising increasing leptin cell signaling comprising inhibiting SOCS-3 activity.

The present invention further comprises a method of treating a mood affective disorder in a mammal comprising inhibiting SOCS-3 activity.

The antagonists/agonists of the present invention can be formulated into compositions with an effective amount of the inhibitor/antagonist/agonist as the active ingredient. An effective amount of a SOCS-3 inhibitor/antagonist is an amount effective to partially or completely inhibit SOCS-3 activity resulting in increased leptin activity. An effective amount of a SOCS-3 agonist is an amount effective to enhance SOCS-3 activity resulting in a decrease of leptin activity. Methods to evaluated leptin activity, such as monitoring food intake, energy expenditure, weight gain/loss, reproductive function and neuroendocrine function are well-known to those of skill in the art. It will be appreciated that the actual effective amounts of the inhibitor/antagonist/agonist in a specific case will vary according to the specific compound being utilized, the particular composition formulated, the mode of administration and the age, weight and condition of the mammal, for example. Dosages for a particular mammal can be determined by one of ordinary skill in the art using conventional considerations, (e.g. by means of an appropriate, conventional pharmacological protocol).

Such compositions can also comprise a pharmaceutically acceptable carrier, and are referred to herein as pharmaceutical compositions. The compositions of the present invention can be administered intravenously, parenterally, orally, by transdermal patch, by inhalation or by suppository. The inhibitor/antagonist/agonist composition may be administered in a single dose or in more than one dose over a period of time to achieve a level of inhibitor/antagonist/agonist which is sufficient to confer the desired effect.

Suitable pharmaceutical carriers include, but are not limited to water, salt solutions, alcohols, polyethylene glycols, gelatin, carbohydrates such as lactose, amylose or starch, magnesium stearate, talc, silicic acid, viscous paraffin, fatty acid esters, hydroxymethylcellulose, polyvinyl pyrolidone, etc. The pharmaceutical preparations can be sterilized and desired, mixed with auxiliary agents, e.g., lubricants, preservatives, stabilizers, wetting agents, emulsifiers, salts for influencing osmotic pressure, buffers, coloring, and/or aromatic substances and the like which do not deleteriously react with the active compounds. They can also be combined where desired with other active agents, e.g., enzyme inhibitors, to reduce metabolic degradation.

For parenteral application, particularly suitable are injectable, sterile solutions, preferably oily or aqueous solutions, as well as suspensions, emulsions, or implants, including suppositories. Ampoules are convenient unit dosages.

The inhibitors/antagonists/agonists of the present invention can be administered to an individual mammal in need of such treatment, in conjunction with an agent or agents that allow the inhibitor to pass through the blood brain barrier. The inhibitor/antagonist/agonist and the agent can be administered simultaneously or sequentially. Such agents are known in the art, such as those described in U.S. Pat. Nos. 5,112,596; 5,268,164; 5,686,416 and 5,506,206; the teachings of which are incorporated herein by reference in their entirety.

The following Examples are offered for the purpose of illustrating the present invention and are not to be construed to limit the scope of this invention.

### EXAMPLES

### Example 1

### Quantification of CIS and SOCS mRNAs by RT-PCR in ob/ob Mice After Leptin Administration

Ad libitum fed male ob/ob mice (Jackson Laboratories, Bar Harbor, Me.) aged 7-8 weeks were injected intraperitoneally with 100 μg recombinant mouse leptin, (from Eli Lilly, Indianapolis, Ind.) or saline. Two hours later, the mice were decapitated, the skull was reflected from the brain, and hypothalami were isolated by snap freezing in liquid nitrogen. Samples of cerebellum, kidney and liver were also taken. Total RNA from the various tissues was isolated using the RNA-STAT-60 reagent as described by the manufacturer (TEL-TEST, Inc., Friendswood, Tex.). Total RNA purification and subsequent cDNA synthesis was done in parallel from all tissue samples. The CDNA was synthesized from 1.0 μg of total RNA by using dT-oligonucleotides and the Advantage RT-PCR kit from Stratagene (La Jolla, Calif.). The final volume of the cDNA samples was 100 μ. The following primers were used for specific PCR amplification of mouse CIS-1, mouse SOCS-1, mouse SOCS-2 and mouse SOCS-3:

| | | | |
|---|---|---|---|
| CIS-1A: | 5′-ctggagctgcccgggccagcc-3′, | 400 bp (GenBank Acc. Number D31943); | SEQ ID NO:1 |
| CIS-1B: | 5′-caaggctgaccacatctggg-3′; | | SEQ ID NO:2 |
| SOCS-1A: | 5′-ccactccgattaccggcgcatc-3′, | 350 bp (GenBank Accession Number U88325); | SEQ ID NO:3 |
| SOCS-1B: | 5′-gctcctgcagcggccgcacg-3′; | | SEQ ID NO:4 |
| SOCS-2A: | 5′-aagacgtcagctggaccgac-3′, | 300 bp (GenBank Acc. Number U588327); | SEQ ID NO:5 |
| SOCS-2B: | 5′-tcttgttggtaaaggcagtccc-3′; | | SEQ ID NO:6 |
| SOCS-3A: | 5′-accagcgccacttcttcacg-3′, | 450 bp (GenBank Acc. Number U88328); | SEQ ID NO:7 |
| SOCS-3B: | 5′-gtggagcatcatactgatcc-3′. | | SEQ ID NO:8 |

Each 50 μl PCR reaction was carried out with 5.0 μl of CDNA as template. The assay conditions were: 10 mM Tris-HCl (pH 8.8), 50 mM KCl, 1.5 mM MgCl,. 0.O1% gelatin 0.2 mM dNTPs. 20 pmol of each primer. 2.5 units of Taq polymerase (Stratagene) and 1.0 μl of ³²P-dCTP (29.6 TBq/mmol. 370 MBq/ml)(NEN, Boston, Mass.). The mixture was overlaid with 25 μl of mineral oil, and after initial denaturation at 96° C. for 3 min the samples were subjected to 24-32 cycles of amplification: denaturation at 95° C. for 1 min, annealing at 60° C. for 1 min, and extension at 72° C. for 45 seconds. Ten μl of the reaction were then combined with 5 μg of sequencing stop solution (Amersham International, Buckinghamshire, UK) and heated to 85° C. for five minutes before loading 5 μl onto a 4% urea-acrylamide gel (38×31×0.03 cm). Electrophoresis was carried out at 60 W of constant power four hours, before the gels were transferred to filter paper, dried and finally subjected to ³²P quantification by Phosphorimager analysis (Molecular Dynamics).

Preliminary PCR experiments showed that the rate of amplification was linear for CIS-1, SOCS-1 and SOCS-3 when applying less than 30 PCR-cycles. The amplification rate of SOCS-2 was linear for 27 cycles, after which non-linear amplification appeared. We chose 25 cycles of PCR amplification for quantification of CIS-1, SOCS-1, SOCS-2 and SOCS-3. PCR reactions were spiked with ³²P-dCTP and assembled in parallel for each cDNA and subjected to PCR amplification under the above conditions of limiting number of cycles. PCR products were then separated on denaturing acrylamide gels and finally subjected to autoradiography.

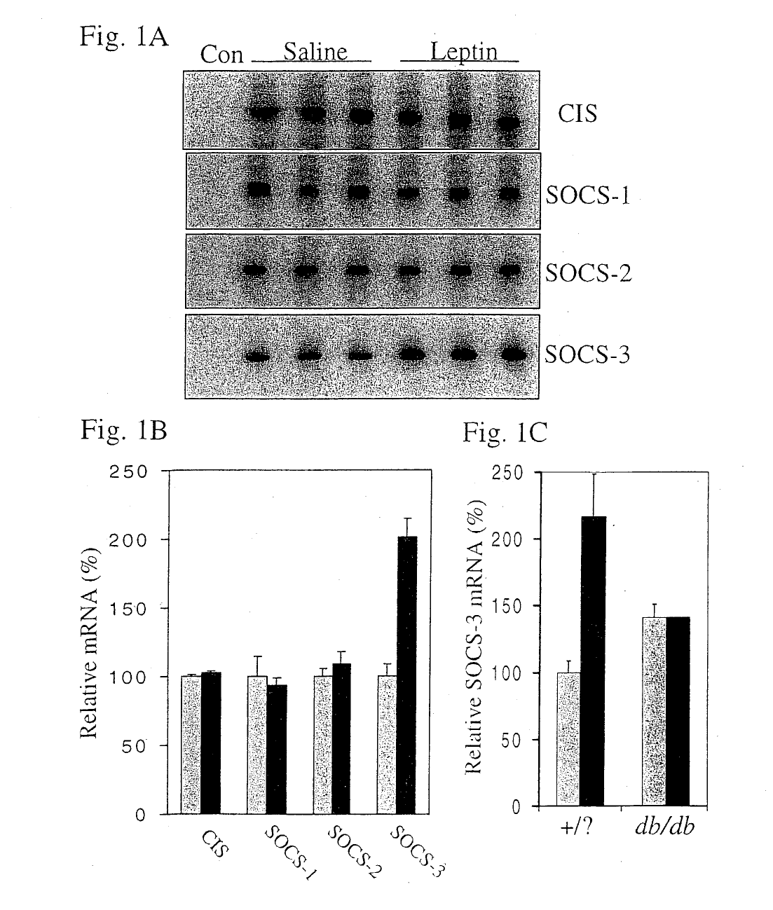

Ad libitum fed male ob/ob mice aged 7-8 weeks were injected intraperitoneally (ip) with 100 μg recombinant mouse leptin, or saline. Two hours later, total RNA was purified from hypothalami, and quantitative RT-PCR for CIS, SOCS-1, SOCS-2 and SOCS-3 mRNAs was performed. Leptin treatment caused a 2.0 fold increase in SOCS-3 mRNA, while no effect on CIS, SOCS-1, or SOCS-2 mRNA levels were detected (FIGS. 1A and 1B). A similar effect on SOCS-3 mRNA was seen 1 or 3 hours after leptin administration (data not shown). No effect of leptin on CIS, SOCS-1, SOCS-2 or SOCS-3 mRNA was detected in cerebellum, kidney or liver (data not shown). To determine whether the effect of leptin on hypothalamic SOCS-3 mRNA was mediated by the long form of the leptin receptor, a similar experiment was performed in db/db mice and control littermates. Leptin increased SOCS-3 mRNA 2.2 fold in hypothalamus of control mice (+/?), while no effect of leptin was detected in db/db mice.

### Example 2

### Localization of SOCS-3 mRNA by in Situ Hybridization in the Rodent Brain After in Vivo Leptin Administration

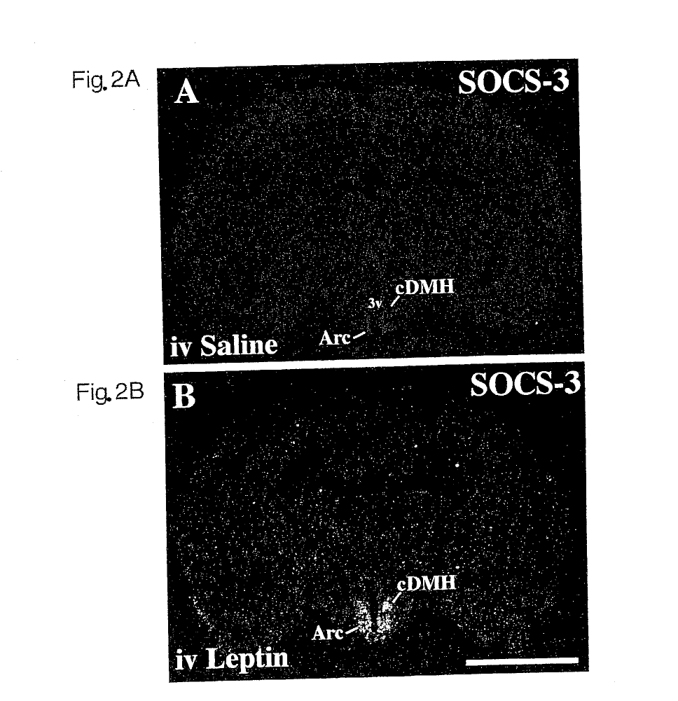

In order to localize the specific anatomic regions of the hypothalamus and other parts of the brain in which leptin affects SOCS-3 mRNA levels, ³⁵S-labeled RNA antisense probe was generated. The SOCS-3A and SOCS-3B primers from above were used amplify a 450 base pair fragment of the mouse SOCS-3 CDNA. The PCR products were cloned into pCR2.1 (Invitrogen, Carlsbad, Calif.) according to the manufactures recommendations. The orientation of the cloned CDNA was verified by sequencing using standard double-stranded plasmid techniques. For generation of sense ³⁵S-labeled RNA, the plasmid was linearized by digestion with BamHI, and subjected to in vitro transcription with T7 polymerase according to the manufactures protocols (Promega). In situ hybridization histochemistry was conducted according to methods well known in the art (Simmons). Tissue sections of mouse and rat brain were mounted onto slides, air dried, and stored in desiccated boxes at −20° C. Prior to hybridization, the slides were immersed in 10% neutral buffered formalin, incubated in 0.001% proteinase K (Boehringer Mannheim) for 30 min., then in 0.025% acetic anhydride for 10 min., and dehydrated in ascending concentrations ethanol. The RNA probes were then diluted to 10⁶ cpm/ml in hybridization solution of 50% formamide, 10 mM Tris-HCl, pH 8.0, 5 mg tRNA, 10 mM dithiothreitol, 10% dextran sulfate, 0.3 M NaCl, 1 mM EDTA, pH 8, and 1× Denhardt's solution (Sigma). Hybridization solution and a glass coverslip was applied to each slide and sections were then incubated for 12-16 hours at 56° C. The coverslips were removed and the slides washed 4 times with 4×SSC. Sections were then incubated in 0.002% RNAase A (Boehringer Mannheim) with 0.5 M NaCl, 10 mM Tris-HCl, pH 8, and 1 mM EDTA, for 30 min. at 37° C. Sections were rinsed in decreasing concentrations of SSC containing 0.25% DTT: 2× at 50° C. for 1 hour, 0.2× at 55° C. for 1 hour, and 0.2× for 1 hour at 60° C. Sections were next dehydrated in graded ethanol (50, 70, 80, and 90%) containing 0.3 M NH₄OAc followed by 100% ethanol. Slides were air dried and placed in X-ray film cassettes with BMR-2 film (Kodak) for 3-5 days. Slides were then dipped in NTB2 photographic emulsion (Kodak), dried and stored with desiccant in foil-wrapped slide boxes at 4° C. for 2-3 weeks. Slides were developed with D-19 developer (Kodak), counterstained with thionin, dehydrated in graded ethanol, cleared in xylene, and coverslipped with Permaslip. Sections were analyzed with a Zeiss Axioplan light microscope using brightfield and darkfield optics. Photomicrographs were produced by capturing images with a digital camera (Kodak, DCS) mounted directly on the microscope and an Apple Macintosh Power PC computer. Image editing software (Adobe Photoshop) was used to combine photomicrographs into plates and figures were printed on a dye sublimation printer (Kodak 8600). Only the sharpness, contrast, and brightness were adjusted. The results are shown in FIG. 2. In brain sections from normal rats fed ad libitum and given a single intravenous injection of recombinant leptin (1 μg/g body weight), strong specific hybridization was detected in the arcuate nucleus (Arc) and the dorsomedial hypothalamic nucleus (DMH), as compared to saline injected rat brain sections (FIGS. 2B and 2A, respectively). In other regions of the brain, including the cerebellum, no specific hybridization signals were detected.

### Example 3

### SOCS-3 Inhibits Leptin Induced Transcriptional Activation in CHO Cells

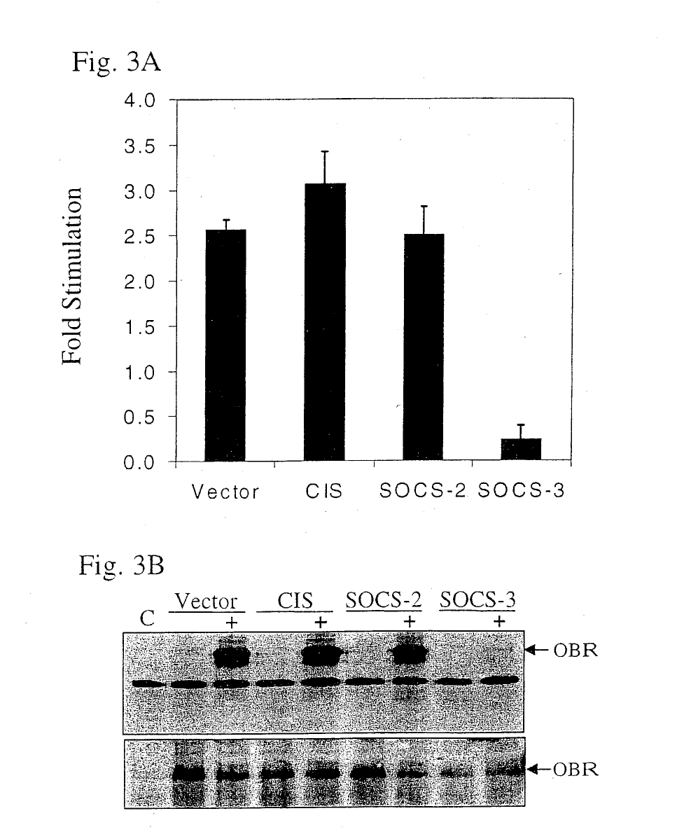

SOCS-3 was tested for its ability to inhibit leptin induced transcriptional activation. erg-1 is an immediate early gene induced upon leptin stimulation. erg-1-luc is a reporter construct expressing the promoter elements of erg-1 fused to the luciferase gene. CHO cells were transiently transfected the leptin receptor erg-1-luc together with either alone or with CIS-1, SOCS-2 or SOCS-3. As shown in FIG. 3A, SOCS-3, but not CIS-1 or SOCS-2 blocked leptin-induced activation of the erg-1 luciferase reporter construct while serum-induced erg-1 gene transcription was unaffected by expression of SOCS-3.

### Example 4

### Localization of SOCS-3 mRNA by in Situ Hybridization in the “Agouti” Mouse

The Agouti (or lethal yellow, A^{y}/a) mouse is an autosomal dominant murine obesity model. Obesity in these mice is accompanied by increased linear growth and altered hair pigmentation. Like other non ob or db mouse models of obesity, Agouti mice have elevated levels of leptin and are refactory to leptin treatment either intravenously or injected directly into brain tissue. The disorder is caused by ectopic and unregulated expression of agouti, a protein normally restricted to hair follicles, where it affects pigmentation by antagonizing melanocyte stimulating hormone (—MSH). Agouti also antagonizes MC4 receptors, whose expression is largely restricted to the brain. SOCS-3 expression was localized in brain tissue of Agouti mice following the methods described in Example 2. Specific hybridization was detected in the arcuate nucleus, while no specific hybridization signals were detected in other regions of the brain. This data supports the theory that expression of SOCS-3 plays a role in the desensitization of these animals to leptin signaling and hence is an important factor in the loss of weight control in these animals.

### Example 5

### Suppression of Leptin Receptor Signaling by SOCS-3 in Mammalian Cell Lines

SOCS-3 was tested for its effect on leptin receptor signaling in mammalian cell lines. COS-1 cells were grown in Dulbecco's modified Eagle's medium (DMEM. low glucose) supplemented with 10% fetal calf serum (FCS), 100 units/ml penicillin and 10 μg/ml streptomycin at 37° C. in 5% CO₂. CHO cells were grown in HAM's F12 medium supplemented with 10% FCS, 100 units/ml penicillin, and 10 μg/ml streptomycin. In all experiments including JAK cDNA, the amount of transfected JAK cDNA was 1/10 of the total amount of DNA transfected. For Western blotting experiments, cells were grown in 10 cm dishes and transfected using 80 μl of Lipofectamine and a total of 20 μg of plasmid DNA. Cells were serum-deprived for 12-15 h prior to stimulation with hormones. Cells were harvested 48 hours post transfection. For Western blotting experiments, cells were rinsed in ice-cold phosphate-buffered saline, and scraped into 1000 μl of ice-cold lysis buffer B (1% Nonidet P-40, 0.5% Triton X-100, 10% glycerol, 150 mM NaCl, 2 mM Na₃V0₄, 20 mM NaF, 1 mM phenylmethylsulfonyl fluoride, 5 μg/ml leupeptin, 5 μl/ml aprotinin, 50 mM Tris-HCl, pH 7.4). Lysates were finally clarified by centrifugation at 23,000 g for 15 min. and the supernatant immunoprecipitated as described below. Immunoprecipitations were performed at 4° C. by incubating clarified cell extracts with the 12CA5 or OBR antibodies and protein A-agarose beads (1:15 dilution of a 50% slurry in 1% Nonidet P-40, 0.5% Triton X-100, 10%, glycerol, 150 mM NaCl, 50 mM Tris-HCI, pH 7.4) on a rotating wheel overnight. The agarose beads were pelleted by low speed centrifugation and washed 3 times with 1 ml of ice-cold lysis buffer B. For immunoblotting, proteins were boiled for 5 min. and subjected to SDS-PAGE, followed by transfer of the resolved polypeptides to nitrocellulose membranes. The membranes were blocked with 10% nonfat dried milk in Towbin buffer (20 mM Tris-HCl, pH 7.4, 150 mM NaCl, 0.05% Tween 20) for 2 h at room temperature and then incubated with antibodies in 5% milk for 12-15 h at 4° C. After removal of unbound antibodies by three washes each for 20 min in Towbin buffer, membranes were incubated with horseradish peroxidase-conjugated anti-rabbit or anti-mouse immunoglobulin (1:1000) in 2.5% milk for 1.5 h at room temperature and washed five times in Towbin buffer. The targeted proteins were detected using enhanced chemiluminescence (ECL) as described by the manufacturer (Amersham International, Buckinghamshire, UK). Stripping of nitrocellulose membranes was done by soaking membranes in 1% SDS, 70 mM Tris-HCI, pH 6.8 and 0.1% mercaptoethanol at 50° C. for 30 minutes with slow agitation.

### Leptin Receptor and Stat3 Phosphorylation

COS-1 cells were transiently co-transfected with expression vectors for mouse OBR1 and JAK2, together with either pcDNA3, or HA-tagged CIS-1, SOCS-2, SOCS-3 in PCDNA3 (Invitrogen).

The intact coding region of CIS was cloned by combining an EST (TIGR clone ID 104844, provided by Damien Dunnington, SmithKline Beecham Pharmaceuticals) with a 5′RACE product derived from human skeletal muscle mRNA (Clontech, Palo Alto, Calif.). The intact coding region of SOCS-2 was cloned by combining an EST (IMAGE clone ID 131550, WashU-Merck EST project) with a 5′RACE product derived from human skeletal muscle Marathon cDNA (Clontech, Palo Alto, Calif.). The intact coding region of SOCS-3 was cloned by PCR amplification from Balb/c mouse genomic DNA (Sigma). A tandem hemagglutinin tag (HA) as fused to the C-terminal ends of all clones, before subcloning into the mammalian expression vector pcDNA3 (Invitrogen, Carlsbad, Calif.). All clones were verified by sequencing.

Forty-eight hours post transfection including 15 hours of serum starvation, cells were either treated or not with 100 nM leptin for 10 minutes. Western blotting of leptin receptor immunoprecipitates with anti-pY antibodies demonstrated that SOCS-3 completely blocked leptin induced leptin receptor tyrosine phosphorylation, while CIS-1 or SOCS-2 had no effect (FIG. 3B, top panel). SOCS-3 was tested for its ability to block downstream signaling by measuring STAT3 tyrosine phosphorylation in transfected COS-1 cells. SOCS-3 also completely blocked leptin induced STAT3 phosphorylation, while CIS-1 and SOCS-2 were without effect.

### JAK2 Phosphorylation

CHO cells were transfected with OBR1 and JAK2 together with either empty vector, CIS-HA, SOCS-2-HA, or SOCS-3-HA expression vectors.. Forty-eight hours post transfection, including 15 hours of serum starvation, cells were either treated or not with 100 nM mouse leptin for 5 min. JAK2 immunoprecipitates (anti-JAK2 antibodies were from UBI) were subjected to SDS-PAGE and Western blotting with antiphosphotyrosine antibodies (4G10, UBI) or anti-JAK2 antibodies (Santa Cruz).

SOCS-3, but not CIS or SOCS-2, inhibited leptin tyrosine phosphorylation of JAK2. Expression of CIS-HA, SOCS-2-HA or SOCS-3-HA has no effect on JAK2 protein expression in these cells.

### Example 6

### Leptin-dependent Co-immunoprecipitation of JAK2 with SOCS-3.

COS-1 cells were transfected with expression vectors encoding OBR1 and JAK2, together with either SOCS-2-HA or SOCS-3-HA expression vectors as described in Example 5. Forty-eight hours post transfection, including 15 h of serum starvation, cells were stimulated or not with 100 nM leptin for 5 minutes. HA-immunoprecipitates (anti-HA antibodies were from Babco) were subjected to SDS-PAGE and Western blotting with anti-JAK2 antibodies (Santa Cruz).

JAK2 was co-immunoprecipitated with SOCS-3, but not SOCS-2, in a leptin-dependent manner in transfected COS-1 cells. These data are consistent with results published earlier on SOCS-1 in transfected 293 cells (Endo et al., 1997). These results, together with the results from above (FIG. 3), are consistent with the possibility that SOCS-3 inhibits leptin-receptor signal transduction by interacting with JAK2 and subsequently inhibiting its tyrosine-kinase activity as proposed for SOCS-1 by Endo et al., 1997.

### Example 7

### Activation of SOCS-3 mRNA by Leptin in CHO Cells Stably Expressing Leptin Receptor

CHO cells stably expressing either then long OBR1 or short (OBRs) form of the leptin receptor were grown and serum-deprived as described in Example 4. Total RNA was isolated from confluent cells grown in 10 cm dishes using the RNA-STAT method as described in Example 1. Northern blotting was performed according to standard procedures (Sambrook et al., 1989), and probed with a ³²P-labelled DNA probe (Gibco-BRL random labelling kit) encompassing the coding region of the mouse SOCS-3 gene.

Serum induces SOCS-3 mRNA in both CHO—OBR and CHO—OBRs cells. Leptin induced SOCS-3 mRNA levels after 1 hour of treatment in CHO-OBR1, but not in CHO—OBRs. After 2 and 4 hours of leptin treatment, SOCS-3 mRNA levels return to baseline in the CHO—OBR1 cells. Therefore leptin has the capability to activate endogenous SOCS-3 gene-expression in cells expressing the long form of the leptin receptor consistent with leptin directly activating SOCS-3 mRNA in neurons expressing OBR1. CIS and SOCS-2 mRNA were not induced by leptin in the two cell lines.

### Example 8

### Leptin Pretreatment of CHO—OBR1 Cells Causes Leptin-Resistance in Proximal Leptin-receptor Signaling

CHO—OBR1 cells were tested for leptin resistance under conditions where endogenous SOCS-3 protein levels are elevated. CHO—OBR1 cells were stimulated with leptin for 1 hour and then washed to remove leptin from the medium. At different times after the leptin pretreatment, freshly applied leptin was tested for the ability to induce intracellular signaling. As demonstrated by Northern blotting, leptin was unable to induce SOCS-3 mRNA for up to 24 hours after leptin pretreatment. On the other hand, in leptin-pretreated cells, fetal calf serum retained the ability to induce SOCS-3 mRNA, suggesting that leptin pretreatment of CHO—OBR1 cells causes leptin-resistant signaling at a step upstream of the socs-3 gene.

Because induction of socs genes by cytokines has been reported to require STAT activation, STAT DNA-binding activities by leptin in CHO—OBR1 cells was measured using an electrophoretic-mobility-shift-assay (EMSA) specific for STAT1 and STAT3 using methods well known in the art. Leptin rapidly induced activation of STAT DNA-binding activities with maximal levels detected after ˜5 minutes of leptin treatment. However, as demonstrated by EMSA, leptin was unable to activate STAT for up to 24 hours after leptin pretreatment. Yet in the same leptin-pretreated cells, TNF-a retained a full ability to activate STAT, suggesting that leptin pretreatment of CHO cells causes blockade of leptin signaling at a step upstream of STAT activation.

Because proximal leptin signaling involves tyrosine phosphorylation by JAK kinases of the leptin receptor, leptin pretreatment of CHO—OBR1 cells was tested for the ability to inhibit subsequent stimulation of leptin receptor phosphorylation. Pretreatment with 3 or 100 nM leptin for 1 hour blocked the ability of fresh leptin to induce receptor phosphorylation. Binding of tracer leptin was not significantly affected by prior leptin treatment as measured 1.5-24 hours after leptin pretreatment; therefore, the reduced level of leptin receptor phosphorylation was not due to downregulation of the leptin receptor itself. Collectively, these data demonstrate that leptin pretreatment of CHO—OBR1 cells results in blockade of proximal leptin signaling without affecting surface leptin receptor expression.

### Equivalents

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A method of modulating leptin cell signaling activity comprising modulating SOCS-3 activity.

2. The method of claim 1 wherein SOCS-3 activity is inhibited, resulting in increased leptin cell signaling activity.

3. The method of claim 2 wherein the expression of SOCS-3 protein is inhibited, comprising introducing a nucleotide construct comprising a polynucleotide wherein the polynucleotide prevents transcription of SOCS-3 DNA or
introducing a nucleotide construct comprising a polynucleotide encoding SOCS-3 antisense mRNA into a cell, wherein the antisense SOCS-3 mRNA binds to endogenous SOCS-3 mRNA in the cell, thereby inhibiting expression of SOCS-3 protein or
introducing a nucleotide construct comprising a polynucleotide encoding a polypeptide comprising modified SOCS-3 into a cell, wherein the modified SOCS-3 polypeptide is a competitive inhibitor of endogenous SOCS-3 thereby inhibiting SOCS-3 activity or
introducing a SOCS-3 inhibitor into a cell wherein the inhibitor interferes with the interaction of SOCS-3 with a SOCS-3 target protein.

4. The method of claim 3 wherein the SOCS-3 target protein is JAK2.

5. The method of claim 3 wherein the SOCS-3 inhibitor is selected from the group consisting of: polypeptides, peptides, peptide mimetics, organic molecules, antibodies and antibody fragments wherein the inhibitor interacts with SOCS-3 or the SOCS-3 target protein, thereby interfering with the interaction of SOCS-3 with the SOCS-3 target protein, resulting in the inhibition of SOCS-3 activity and an increase in leptin-induced cell signaling activity.

6. The method of claim 1 wherein SOCS-3 activity is increased resulting in decreased leptin cell signaling activity.

7. The method of claim 6 wherein the expression of SOCS-3 protein is increased, resulting in increased SOCS-3 activity and a decrease in leptin cell signaling activity.

8. The method of claim 7 comprising introducing into a cell a nucleotide construct, comprising a polynucleotide encoding a SOCS-3 polypeptide or a modified SOCS-3 polypeptide.

9. A SOCS-3 inhibitor comprising a molecule selected from the group consisting of polypeptides, peptides, antibodies, antibody fragments, peptide mimetics, small organic molecules and nucleic acids.

10. A cell line expressing SOCS-3, a cytokine receptor and a reporter gene construct wherein transcription of the reporter gene is inhibited by SOCS-3 mRNA induction.

11. The cell line of claim 10 wherein the reporter gene construct contains SOCS-3 promoter elements.

12. The cell line of claim 10 wherein the cytokine receptor is the leptin receptor long form.

13. A method for identifying inhibitors of SOCS-3 activity comprising the steps of:
a) culturing the cells of claim 10 under conditions suitable for growth;
b) contacting the cells of step a) with an organic molecule library comprising candidate SOCS-3 inhibitors or transfecting said cells with a cDNA expression library comprising DNA encoding candidate SOCS-3 inhibitors;
c) contacting the cells of step b) with leptin;
d) selecting the cells of step c) having increased reporter gene activity; and
e) identifying the organic molecule or cDNA that had contacted the cells selected in step d).

14. A cytokine dependent cell line wherein the cell line stably expresses the leptin receptor long form and SOCS-3.

15. The cell line of claim 14 wherein the cytokine is IL-3.

16. A method for identifying inhibitors of SOCS-3 activity comprising the steps of:
a) culturing the cells of claim 15 in the presence of IL-3 under conditions suitable for growth;
b) removing the cells of step a) from the presence of IL-3;
c) contacting the cells of step b) with an organic molecule library comprising candidate SOCS-3 inhibitors or transfecting said cells with a cDNA expression library comprising candidate SOCS-3 inhibitors;
d) contacting the cells of step c) with leptin;
e) selecting the cells of d), that are capable of proliferating in the presence of leptin; and
f) identifying the organic molecule or cDNA that had contacted the cells selected in e).

17. A SOCS-3 inhibitor identified by the method of claim 13 or claim 16.

18. Use of a leptin cell-signaling enhancer for the manufacture of a medicament or therapeutic agent in an amount effective to cause a reduction in weight or a reduction of food intake in a mammal.

19. Use of a SOCS-3 inhibitor for the manufacture of a medicament or a therapeutic agent in an amount effective to cause a reduction in weight or a reduction of food intake in a mammal.

20. Use of a SOCS-3 inhibitor for the manufacture of a medicament or therapeutic agent, in an amount effective to improve a mood disorder condition in a mammal.
